(19) United States
(12) Patent Application Publication
(10) Pub. No.: US 2010/0003276 A1
(43) Pub. Date: Jan. 7, 2010

(54) Methods for treating anthrax and inhibiting lethal factor

(76) Inventor: Jeffery D. Hermes, Warren, NJ (US)

Correspondence Address: MERCK AND CO., INC, P O BOX 2000, RAHWAY, NJ, 07065-0907, US

(21) Appl. No.: 11/006,335

(22) Filed: Dec. 7, 2004

(51) Int. Cl. A61K 39/385; A61K 39/07; A61P 31/04

(52) U.S. Cl. 424/197.11; 424/236.1

(57) ABSTRACT

This invention relates to a method of inhibiting lethal factor (LF) or for treating anthrax and other conditions related to anthrax infection comprising co-administration of an effective amount of an LF inhibitor and a vaccine to a patient in need of such treatment. Such co-administration unexpectedly provides an effective immune response.

### BACKGROUND OF THE INVENTION

The references cited throughout the present application are not admitted to be prior art to the claimed invention.

Anthrax is a bacterial infection produced by Bacillus anthracis. Bacillus anthracis endospores can enter the body through skin abrasions, inhalation, or ingestion. Bacillus anthracis produces an anthrax toxin that is often lethal. (Dixon et al., (1999) N. Engl. J. Med. 341, 815-26.)

Anthrax toxin consists of three proteins, a receptor-binding component designated protective antigen, and two enzymatic components termed edema factor and lethal factor (“LF”). (Mock et al., (2001) Annu. Rev. Microbiol. 55, 647-71.) Lethal factor is a zinc-dependent metalloprotease that appears to exert toxic affects by cleaving mitogen-activated protein kinase kinases (MKKs). (Vitale et al., (1998) Biochem. Biophys. Res. Commun. 248, 706-11, Vitale et al., (2000) Biochem. J. 352 Pt 3, 739-45, Duesbery et al., (1998) Science 280, 734-7, Duesbery et al., International Publication No. WO 99/50439, International Publication Date Oct. 7, 1999).

Vitale and co-workers have used microsequencing to identify the site in different MKKs that are cleaved by lethal factor. (See Table 1, Vitale et al., (2000) Biochem. J. 352 Pt 3, 739-45.) Lethal factor cleavage of different MKKs occurred within the N-terminal region preceding the kinase domain. Alignment of the sequences flanking the cleavage site revealed some consensus motifs: a hydrophobic residue in position P2 and P1′, and at least one basic residue between P4 and P7. (Vitale et al., (2000) Biochem. J. 352 Pt 3, 739-45.)

Lethal factor has been indicated to cleave synthetic peptides in vitro. (Hammond et al., (1998) Infect. Immun. 66, 2374-8.) In vitro cleavage was inhibited by 1,10-phenanthroline or 10 mM EDTA, both of which chelate zinc. Lethal factor also has been shown to suppress the dendritic cells ability to prime T cells (Agrawal, et al, Nature 424, 329-334 (17 Jul. 2003).

Most Bacillus anthracis strains are sensitive to a broad range of antibiotics. The commonly prescribed therapies today are ciprofloxacin, penicillin, or doxycycline. However, the efficacy and side effect profiles of these agents are not ideal.

While antibiotics can kill the bacteria that cause anthrax, the tripartite anthrax toxin continues to damage the body even when the bacteria themselves are dead. Therefore, there still exist the need for new and effective therapies with improved efficacy, little or no side effect and which inhibit the scissor-like ability of lethal factor to snip apart important host molecules.

This invention relates to treatment therapies involving the co-administration of a lethal factor inhibitor and vaccine. This vaccine approach functions to build up host immunity to anthrax components. This is significantly important since ungerminated spores may persist in parts of the body for months or longer. As a result of the claimed treatment therapy, the fortified host immune response will persist after pharmaceutical agents, such as antibiotics, have been terminated.

### SUMMARY OF THE INVENTION

This invention relates to a method of inhibiting lethal factor or for treating anthrax and other conditions related to anthrax infection comprising co-administration of an effective amount of an LF inhibitor and a vaccine to a patient in need of such treatment. Such co-administration provides a unique and effective immune response for the treatment of anthrax and for inhibiting lethal factor. The invention further relates to a method of inhibiting lethal factor or for treating anthrax and other conditions related to anthrax infection comprising co-administering to a patient in need thereof a vaccine and a compound of formula I:

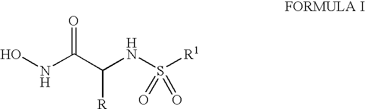

or a pharmaceutically acceptable salt, enantiomer, diastereomer or in vivo hydrolysable ester or mixture thereof, wherein,
R¹ represents C₆₋₁₀ aryl, C₅₋₁₀ heteroaryl or C₅₋₁₀ heterocyclic, said aryl, heteroaryl and heterocyclyl optionally substituted with 1 to 3 groups of R^{a}

R^{a} represents C₁₋₆ alkyl, halogen, OH, aryl(C₁₋₆)alkyl, (C₁₋₆)alkoxy, (C₁₋₆)alkoxy(C₁₋₆)alkyl, halo(C₁₋₁₆)alkyl, nitro, amino, mono- or di-N—(C₁₋₁₆)alkylamino, acylamino, acyloxy, carboxy, carboxy salts, carboxy esters, carbamoyl, mono- and di-N—(C₁₋₆)alkylcarbamoyl, (C₁₋₆) alkoxycarbonyl, aryloxycarbonyl, ureido, guanidino, sulphonylamino, aminosulphonyl, (C₁₋₆)alkylthio, (C₁₋₆)alkylsulphinyl, (C₁₋₆)alkylsulphonyl, heterocyclyl, heterocyclyl(C₁₋₆)alkyl; and

R represents C₁₋₈ alkyl, C₃₋₁₀ cycloalkyl, C₃₋₁₀ heterocycloalkyl, C₅₋₁₀ heteroaryl, or C₅₋₁₁ heterocyclyl, said heteroaryl and heterocyclyl optionally substituted with 1 to 3 groups of R^{a} and said alkyl, optionally substituted with 1-3 groups selected from the group consisting of aryl, heterocyclyl, (C₁₋₆)alkylthio, cyano, heteroaryl, guanidino, ((1-aminoethyl)carbonyl)amino, ((aminomethyl)carbonyl)amino, ((2-amino)prop-2-yl) carbonyl)amino, acetamido, 4-(aminomethyl)phenyl, thio, t-butyl sulfonyl, (C₂₋₆)alkenylthio, (C₂₋₆)alkynylthio, amino, mono- or di-(C₁₋₆)alkylamino, arylthio, heterocyclylthio, (C₁₋₆)alkoxy, aryl(C₁₋₆)alkoxy, aryl(C₁₋₆)alkylthio, cycloalkyl, cycloalkenyl, carboxy and esters thereof, hydroxy and halogen.

This and other aspects of the invention will be realized upon inspection of the invention as a whole.

### DETAILED DESCRIPTION OF THE INVENTION

The present invention is directed to a method for treating anthrax or inhibiting lethal factor by co-administration, preferably intravenous or intra-muscular, of a composition containing a compound of formula I and a pharmaceutically acceptable carrier and a composition containing a vaccine and a pharmaceutically acceptable carrier.

The vaccines useful for this invention are protective antigen based (PA) vaccines, e.g., purified protein from B. anthracis culture or live-attenuated spore vaccine antitoxin vaccines, such as AVA or any of the more modern, defined PA, capsule-based or a conjugate of PA and capsule-based vaccines. An anthrax vaccine (Bioport Corp.) is available. This vaccine consists of a membrane-sterilized culture filtrate of B. anthracis V770-NP1-R, an avirulent, nonencapsulated strain. The culture filtrate is adsorbed to aluminum hydroxide and formulated with benzethonium chloride (preservative) and formaldehyde (stabilizer). The administration schedule consists of 0.5 ml injected subcutaneously at 0, 2, and 4 weeks, 6, 12, and 18 months, and then annually thereafter. This method of treating anthrax and/or inhibiting lethal factor using the combination of LF inhibitor with any type of anthrax vaccine including, but not limited to, vaccines directed to toxin or capsule antigens is a new and effective treatment therapy.

Another aspect of this invention is a method of inhibiting lethal factor and/or treating anthrax comprising the co-administration of an LF inhibitor, a PA-based vaccine and an antibiotic selected from the group consisting of penicillin, doxycycline, ciprofloxacin, penicillin, tetracycline, chloramphenicol, erythromycin, vancomycin, cefazolin, and aminoglycosides rifampin, vancomycin, clindamycin, imipenem, meropenem, chloramphenicol clarithromycin, azithromycin, ceftriaxone, sulfamethoxazole, and trimethoprim.

The invention is described herein in detail using the terms defined below unless otherwise specified.

When any variable (e.g. aryl, heterocycle, R¹, R etc.) occurs more than one time in any constituent, its definition on each occurrence is independent at every other occurrence. Also, combinations of substituents/or variables are permissible only if such combinations result in stable compounds.

The term “alkyl” refers to a monovalent alkane (hydrocarbon) derived radical containing from 1 to 10 carbon atoms unless otherwise defined. It may be straight, branched or cyclic. Preferred alkyl groups include methyl, ethyl, propyl, isopropyl, butyl, t-butyl, cyclopentyl and cyclohexyl. When the alkyl group is said to be substituted with an alkyl group, this is used interchangeably with “branched alkyl group”.

Preferably, alkenyl is C₂-C₆ alkenyl.

Preferably, alkynyl is C₂-C₆ alkynyl.

Cycloalkyl is a specie of alkyl containing from 3 to 15 carbon atoms, unless otherwise specified, without alternating or resonating double bonds between carbon atoms. It may contain from 1 to 4 rings that are fused. Examples of cycloalkyl groups are cyclopropyl, cyclobutyl, cyclopentyl, cyclohexyl, and cycloheptyl. Heterocycloalkyl is intended to mean cycloalkyl ring groups which consists of carbon atoms and from one to four heteroatoms selected from the group consisting of N, O, and S, and including any bicyclic. Said heterocycloalkyl can optionally be substituted with 1 to 3 groups of R^{a} described herein. Examples of Heterocycloalkyls are oxane, methyloxane, dioxane, pyran, thiolane, piperidine, pyrrolidine, aziridine, azetidine, etc.

Alkoxy refers to C₁-C₆ alkyl-O—, with the alkyl group optionally substituted as described herein. Examples of alkoxy groups are methoxy, ethoxy, propoxy, butoxy and isomeric groups thereof.

Halo is short for halogen and refers to chloride, fluoride, bromide and iodide.

As used herein, “aryl” is intended to mean any stable monocyclic or bicyclic carbon ring of up to 7 members in each ring, wherein at least one ring is aromatic. Examples of such aryl elements include phenyl, naphthyl, tetrahydronaphthyl, indanyl, biphenyl, phenanthryl, anthryl or acenaphthyl.

The term heterocyclyl or heterocyclic, as used herein, represents a stable 5- to 7-membered monocyclic or stable 8- to 11-membered bicyclic heterocyclic ring which is either saturated or unsaturated, and which consists of carbon atoms and from one to four heteroatoms selected from the group consisting of N, O, and S, and including any bicyclic group in which any of the above-defined heterocyclic rings is fused to a benzene ring. The heterocyclic ring may be attached at any heteroatom or carbon atom which results in the creation of a stable structure. A fused heterocyclic ring system may include carbocyclic rings and need include only one heterocyclic ring. The term heterocycle or heterocyclic includes heteroaryl moieties. Examples of such heterocyclic elements include, but are not limited to, azepinyl, benzimidazolyl, benzisoxazolyl, benzofurazanyl, benzopyranyl, benzothiopyranyl, benzofuryl, benzothiazolyl, benzothienyl, benzoxazolyl, chromanyl, cinnolinyl, dihydrobenzofuryl, dihydrobenzothienyl, dihydrobenzothiopyranyl, dihydrobenzothiopyranyl sulfone, 1,3-dioxolanyl, furyl, imidazolidinyl, imidazolinyl, imidazolyl, indolinyl, indolyl, isochromanyl, isoindolinyl, isoquinolinyl, isothiazolidinyl, isothiazolyl, isothiazolidinyl, morpholinyl, naphthyridinyl, oxadiazolyl, 2-oxoazepinyl, oxazolyl, 2-oxopiperazinyl, 2-oxopiperidinyl, 2-oxopyrrolidinyl, piperidyl, piperazinyl, pyridyl, pyrazinyl, pyrazolidinyl, pyrazolyl, pyridazinyl, pyrimidinyl, pyrrolidinyl, pyrrolyl, quinazolinyl, quinolinyl, quinoxalinyl, tetrahydrofuryl, tetrahydroisoquinolinyl, tetrahydroquinolinyl, thiamorpholinyl, thiamorpholinyl sulfoxide, thiazolyl, thiazolinyl, thienofuryl, thienothienyl, and thienyl. An embodiment of the examples of such heterocyclic elements include, but are not limited to, azepinyl, benzimidazolyl, benzisoxazolyl, benzofurazanyl, benzopyranyl, benzothiopyranyl, benzofuryl, benzothiazolyl, benzothienyl, benzoxazolyl, chromanyl, cinnolinyl, dihydrobenzofuryl, dihydrobenzothienyl, dihydrobenzothiopyranyl, dihydrobenzothiopyranyl sulfone, furyl, imidazolidinyl, imidazolinyl, imidazolyl, indolinyl, indolyl, isochromanyl, isoindolinyl, isoquinolinyl, isothiazolidinyl, isothiazolyl, isothiazolidinyl, morpholinyl, naphthyridinyl, oxadiazolyl, 2-oxoazepinyl, oxazolyl, 2-oxopiperazinyl, 2-oxopiperidinyl, 2-oxopyrrolidinyl, piperidyl, piperazinyl, pyridyl, 2-pyridinonyl, pyrazinyl, pyrazolidinyl, pyrazolyl, pyridazinyl, pyrimidinyl, pyrrolidinyl, pyrrolyl, quinazolinyl, quinolinyl, quinoxalinyl, tetrahydrofuryl, tetrahydroisoquinolinyl, tetrahydroquinolinyl, thiamorpholinyl, thiamorpholinyl sulfoxide, thiazolyl, thiazolinyl, thienofuryl, thienothienyl, thienyl and triazolyl.

Preferably, heterocycle is selected from 2-azepinonyl, benzimidazolyl, 2-diazapinonyl, imidazolyl, 2-imidazolidinonyl, indolyl, isoquinolinyl, morpholinyl, piperidyl, piperazinyl, pyridyl, pyrrolidinyl, 2-piperidinonyl, 2-pyrimidinonyl, 2-pyrollidinonyl, quinolinyl, tetrahydrofuryl, tetrahydroisoquinolinyl, and thienyl.

As used herein, “heteroaryl” is intended to mean any stable monocyclic or bicyclic carbon ring of up to 7 members in each ring, wherein at least one ring is aromatic and wherein from one to four carbon atoms are replaced by heteroatoms selected from the group consisting of N, O, and S. Examples of such heterocyclic elements include, but are not limited to, benzimidazolyl, benzisoxazolyl, benzofurazanyl, benzopyranyl, benzothiopyranyl, benzofuryl, benzothiazolyl, benzothienyl, benzoxazolyl, chromanyl, cinnolinyl, dihydrobenzofuryl, dihydrobenzofuranyl, dihydrobenzothienyl, dihydrobenzothiopyranyl, dihydrobenzothiopyranyl sulfone, furyl, imidazolyl, indolinyl, indolyl, isochromanyl, isoindolinyl, isoquinolinyl, isothiazolyl, naphthyridinyl, oxadiazolyl, pyridyl, pyrazinyl, pyrazolyl, pyridazinyl, pyrimidinyl, pyrrolyl, quinazolinyl, quinolinyl, quinoxalinyl, tetrahydroisoquinolinyl, tetrahydroquinolinyl, thiazolyl, thienofuryl, thienothienyl, thienyl and triazolyl.

In one embodiment of this invention R of formula I is a C₁₋₄ alkyl, heterocycloalkyl or heteroaryl and all other variables are as originally described.

In another embodiment of this invention R formula I is a heteroaryl and all other variables are as originally described.

In still another embodiment of this invention R¹ of formula I is a phenyl group optionally substituted with 1-3 groups of R^{a} and R is a heterocycloalkyl, or heteroaryl group.

In still another embodiment of this invention R¹ of formula Ia is a phenyl group optionally substituted with 1-3 groups of R^{a} and R is an alkyl, heterocycloalkyl, or heteroaryl group.

In yet another embodiment of the invention R¹ of formula Ia is a phenyl group substituted with 1 to 3 groups of methoxy, halogen, methyl, ethyl, propyl, butyl, napthyl, 5-(2-pyridyl)thiophen-2-yl or a mixture thereof, and R is an alkyl, heterocycloalkyl or heteroaryl.

Another embodiment of this invention involves the co-administration of a compound of formula I and a PA-based vaccine for the production of a medicament for the treatment or prophylaxis of anthrax and conditions related thereto. Still another embodiment involves the co-administration of a compound of formula I and a PA-based vaccine for the production of a medicament for inhibiting lethal factor. Yet another embodiment involves the co-administration of a compound of formula I, PA-based vaccine and antibiotic selected from the group consisting of penicillin, doxycycline, and ciprofloxacin penicillin, tetracycline, chloramphenicol, erythromycin, vancomycin, cefazolin, and aminoglycosides rifampin, vancomycin, clindamycin, imipenem (used with cilastatin), chloramphenicol clarithromycin, azithromycin, ceftriaxone, sulfamethoxazole, and trimethoprim.

Examples of vaccines useful for this invention are anthrax vaccine (Bioport Corp., Lansing, Mich.), PA vaccine, Bacillus anthracis live spore vaccine, PA toxoid vaccines, Pa producing live vaccines, recombinant anthrax toxin vaccine and the like.

Suitable pharmaceutically acceptable salts of the compounds used in this invention include acid addition salts such as hydrochloride, hydrobromide, citrate, maleate and salts formed with phosphoric and sulphuric acid. In another aspect suitable salts are base salts such as an alkali metal salt for example sodium or potassium, an alkaline earth metal salt for example calcium or magnesium, an organic amine salt for example triethylamine, morpholine, N-methylpiperidine, N-ethylpiperidine, procaine, dibenzylamine, N,N-dibenzylethylamine or amino acids for example lysine. Preferred pharmaceutically acceptable salts are sodium and potassium salts.

In vivo hydrolysable esters are those pharmaceutically acceptable esters that hydrolyze in the human body to produce the parent compound. Such esters can be identified by administering, e.g. intravenously to a test animal, the compound under test and subsequently examining the test animal's body fluids. Suitable in vivo hydrolysable esters for carboxy include C₁₋₆alkoxymethyl esters for example methoxymethyl, C₁₋₆ alkanolyloxymethyl esters for example pivaloyloxymethyl, phthalidyl esters and the additional esters disclosed in U.S. Pat. No. 5,478,820, which is herein incorporated by reference in its entirety.

Compounds useful in this invention are:
N-t-butoxy-2(R)-[(4-fluoro-3-methylphenylsulfonyl)]amino-3-methylbutyramide;
N-hydroxy-2(R)-[(4-fluoro-3-methylphenylsulfonyl)]amino-3-methylbutyramide;
N-t-butoxy-2(R)-[(4-fluoro-3-methylphenylsulfonyl)]amino-2-(4′-tetrahydropyranyl)-acetamide;
N-hydroxy-2(R)-[(4-fluoro-3-methylphenylsulfonyl)]amino-2-(4′-tetrahydropyranyl)-acetamide;
N-hydroxy-2(R)-[(4-fluoro-3-methylphenylsulfonyl)]amino-3-(S)-cyclopropylbutyramide; and pharmaceutically acceptable salts, enantiomers, diastereomers or in vivo hydrolysable esters or mixtures thereof.

Additional compounds useful in this invention are disclosed in Table 1:

TABLE 1

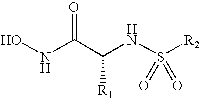

| Example # | R1 | R2 |
|---|---|---|
| 3 | 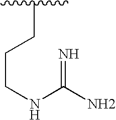 | 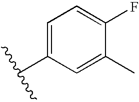 |
| 4 | 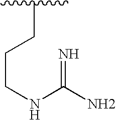 | 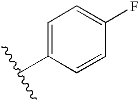 |
| 5 | 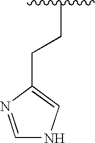 | 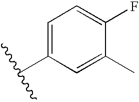 |
| 6 | 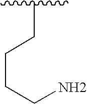 | 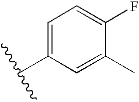 |
| 7 | 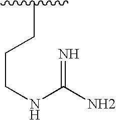 | 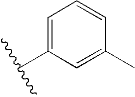 |
| 8 | 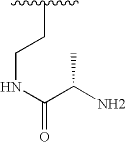 | 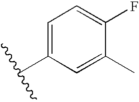 |
| 9 | 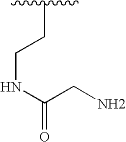 | 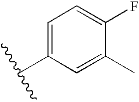 |
| 10 | 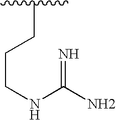 | 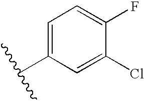 |
| 11 | 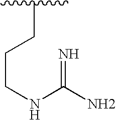 | 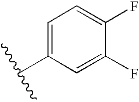 |
| 12 | 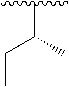 | 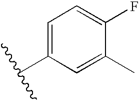 |
| 13 | 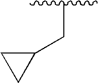 | 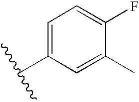 |
| 14 | 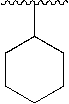 | 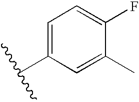 |
| 15 | 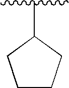 | 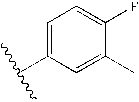 |
| 16 | 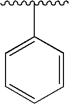 | 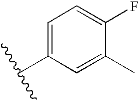 |
| 17 | 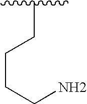 | 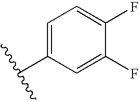 |
| 18 | 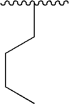 | 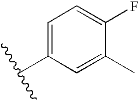 |
| 19 | 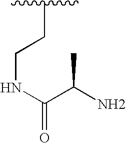 | 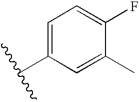 |
| 20 | 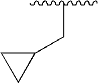 | 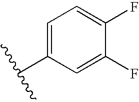 |
| 21 | 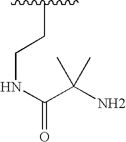 | 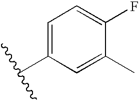 |
| 22 | 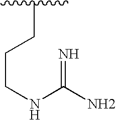 | 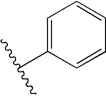 |
| 23 | 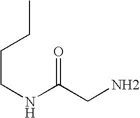 | 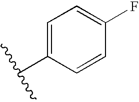 |
| 24 | 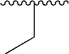 | 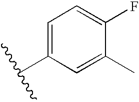 |
| 25 | 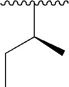 | 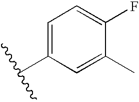 |
| 26 | 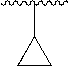 | 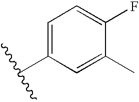 |
| 27 | 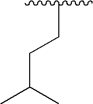 | 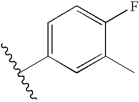 |
| 28 | 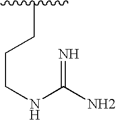 | 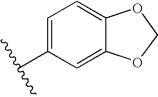 |
| 29 | 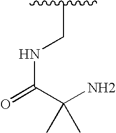 | 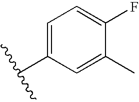 |
| 30 | 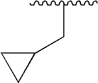 | 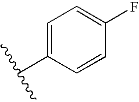 |
| 31 | 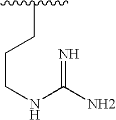 | 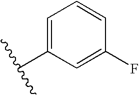 |
| 32 | 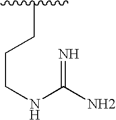 | 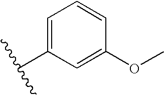 |
| 33 | 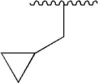 | 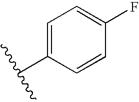 |
| 34 | 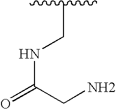 | 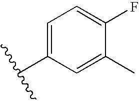 |
| 35 | 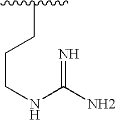 | 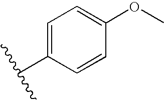 |
| 36 | 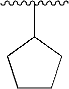 | 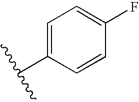 |
| 37 | 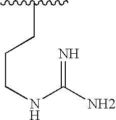 | 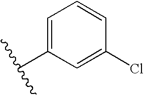 |
| 38 | 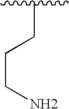 | 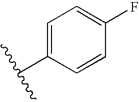 |
| 39 | 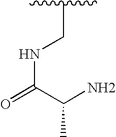 | 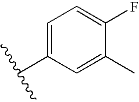 |
| 40 | 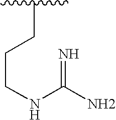 | 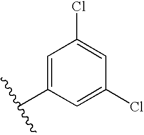 |
| 41 | 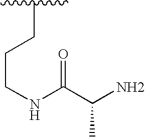 | 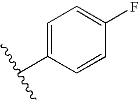 |
| 42 | 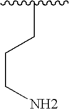 | 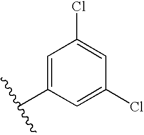 |
| 43 | 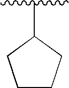 | 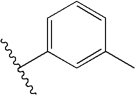 |
| 44 | 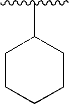 | 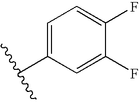 |
| 45 | 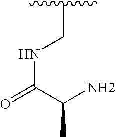 | 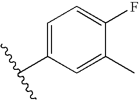 |
| 46 | 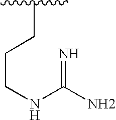 | 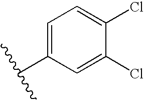 |
| 47 | 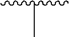 | 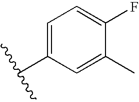 |
| 48 | 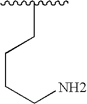 | 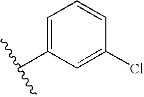 |
| 49 | 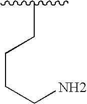 | 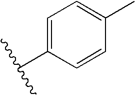 |
| 50 | 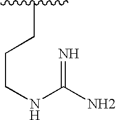 | 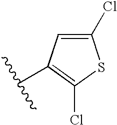 |
| 51 | 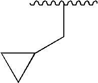 | 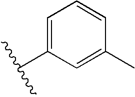 |
| 52 | 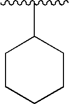 | 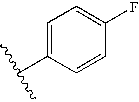 |
| 53 | 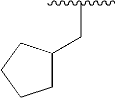 | 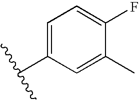 |
| 54 | 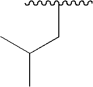 | 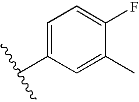 |
| 55 | 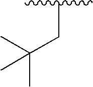 | 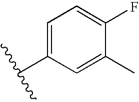 |
| 56 | 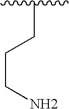 | 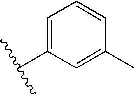 |
| 57 | 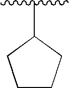 | 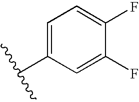 |
| 58 | 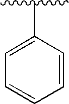 | 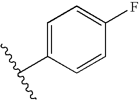 |
| 59 | 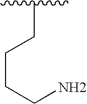 | 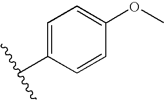 |
| 60 | 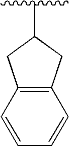 | 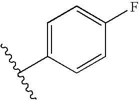 |
| 61 | 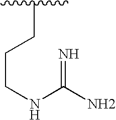 | 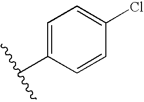 |
| 62 | 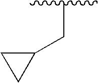 | 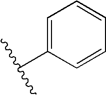 |
| 63 | 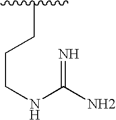 | 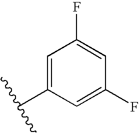 |
| 64 | 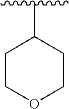 | 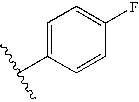 |
| 65 | 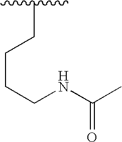 | 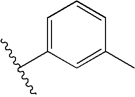 |
| 66 | 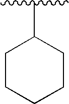 | 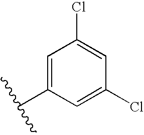 |
| 67 | 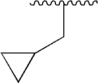 | 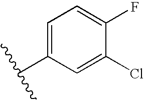 |
| 68 | 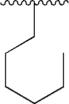 | 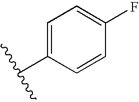 |
| 69 | 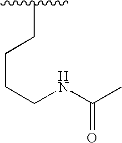 | 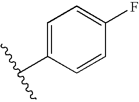 |
| 70 | 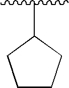 | 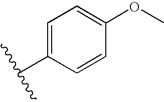 |
| 71 | 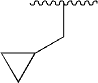 | 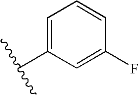 |
| 72 | 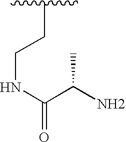 | 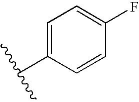 |
| 73 | 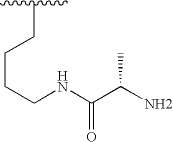 | 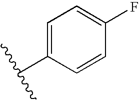 |
| 74 | 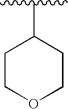 | 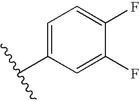 |
| 75 | 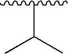 | 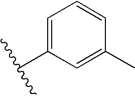 |
| 76 | 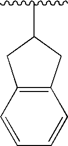 | 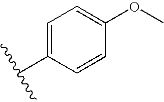 |
| 77 | 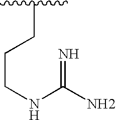 | 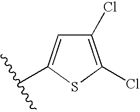 |
| 78 | 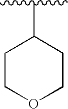 | 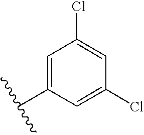 |
| 79 | 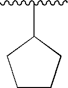 | 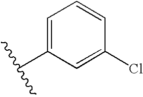 |
| 80 | 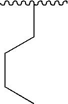 | 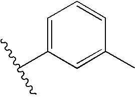 |
| 81 | 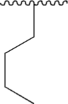 | 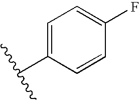 |
| 82 | 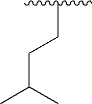 | 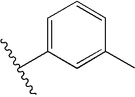 |
| 83 | 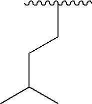 | 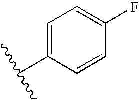 |
| 84 | 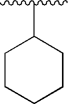 | 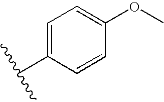 |
| 85 | 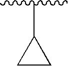 | 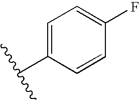 |
| 86 | 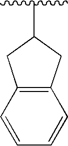 | 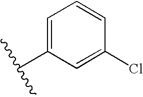 |
| 87 | 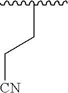 | 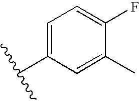 |
| 88 | 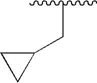 | 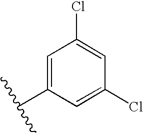 |
| 89 | 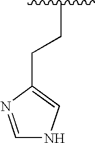 | 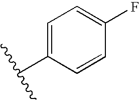 |
| 90 | 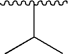 | 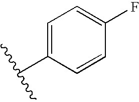 |
| 91 | 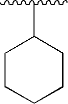 | 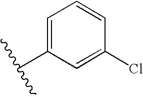 |
| 92 | 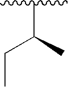 | 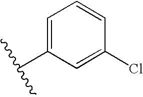 |
| 93 | 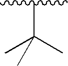 | 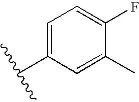 |
| 94 | 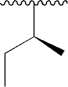 | 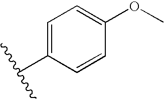 |
| 95 | 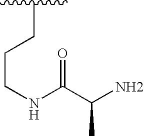 | 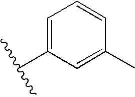 |
| 96 | 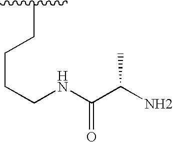 | 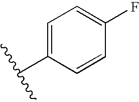 |
| 97 | 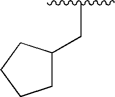 | 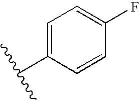 |
| 98 | 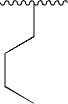 | 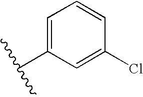 |
| 99 | 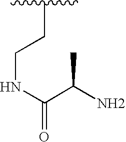 | 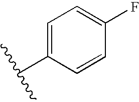 |
| 100 | 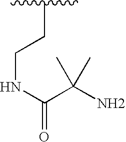 | 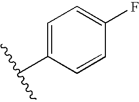 |
| 101 | 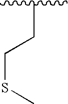 | 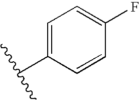 |
| 102 | 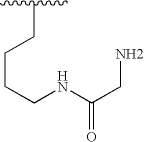 | 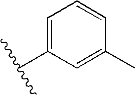 |
| 103 | 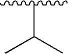 | 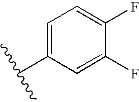 |
| 104 | 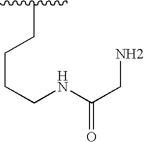 | 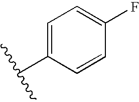 |
| 105 | 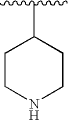 | 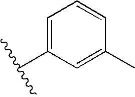 |
| 106 | 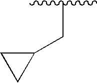 | 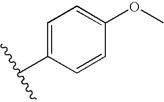 |
| 107 | 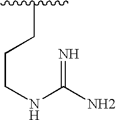 | 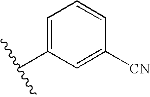 |
| 108 | 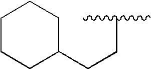 | 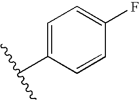 |
| 109 | 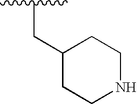 | 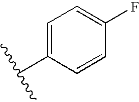 |
| 110 | 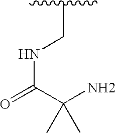 | 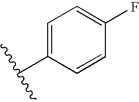 |
| 111 | 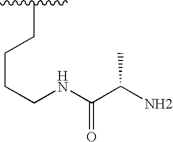 | 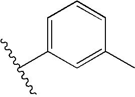 |
| 112 | 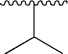 | 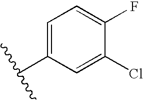 |
| 113 | 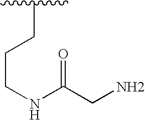 | 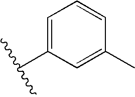 |
| 114 | 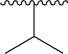 | 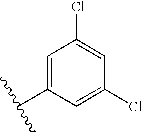 |
| 115 | 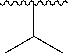 | 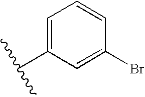 |
| 116 | 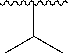 | 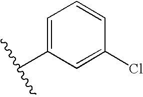 |
| 117 | 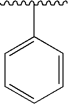 | 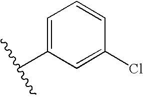 |
| 118 | 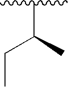 | 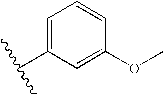 |
| 119 | 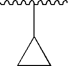 | 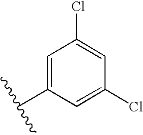 |
| 120 | 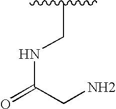 | 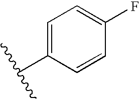 |
| 121 | 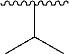 | 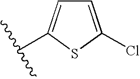 |
| 122 | 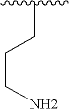 | 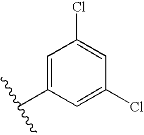 |
| 123 | 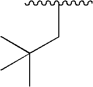 | 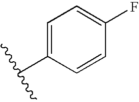 |
| 124 | 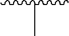 | 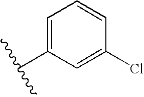 |
| 125 | 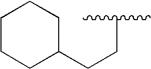 | 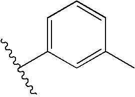 |
| 126 | 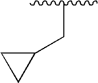 | 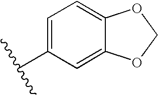 |
| 127 | 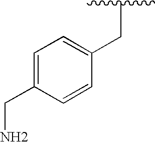 | 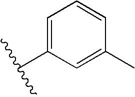 |
| 128 | 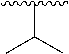 | 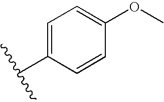 |
| 129 | 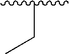 | 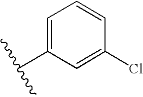 |
| 130 | 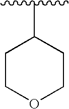 | 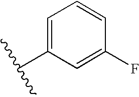 |
| 131 | 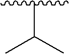 | 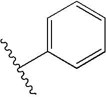 |
| 132 | 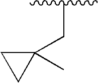 | 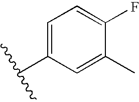 |
| 133 | 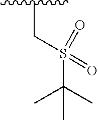 | 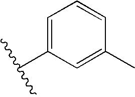 |
| 134 | 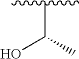 | 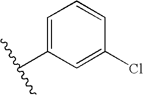 |
| 135 | 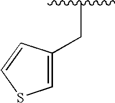 | 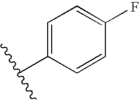 |
| 136 | 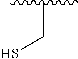 | 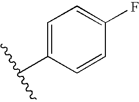 |
| 137 | 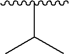 | 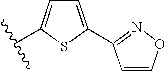 |
| 138 | 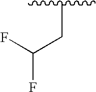 | 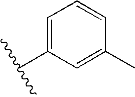 |
| 139 | 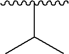 | 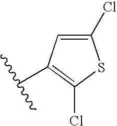 |
| 140 | 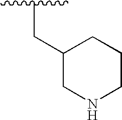 | 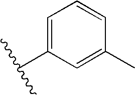 |
| 141 | 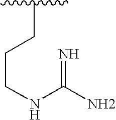 | 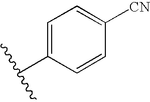 |
| 142 | 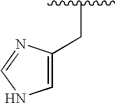 | 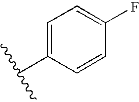 |
| 143 | 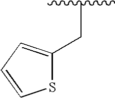 | 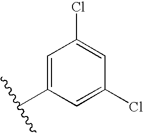 |
| 144 | 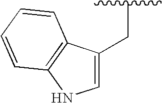 | 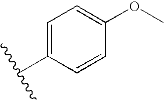 |

and pharmaceutically acceptable salts, enantiomers, diastereomers or in vivo hydrolysable esters or mixtures thereof.

Still other compounds of this invention are disclosed in Table 2:

TABLE 2

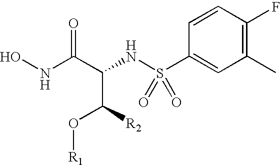

| Example # | R1 | R2 |
|---|---|---|
| 146 | 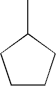 | Me |
| 147 | 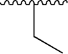 | Me |
| 148 | 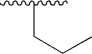 | Me |
| 149 | 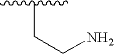 | H |
| 150 | 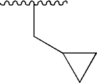 | Me |
| 151 | 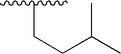 | Me |
| 152 | 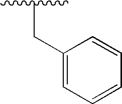 | Me |
| 153 | 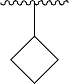 | Me |

and pharmaceutically acceptable salts, enantiomers, diastereomers or in vivo hydrolysable esters or mixtures thereof.

In order to use a compound of formula I or a pharmaceutically acceptable salt, enantiomer, diastereomer or in vivo hydrolysable ester or mixture thereof for the therapeutic treatment of mammals, including humans, in particular in treating anthrax, or inhibiting lethal factor it is normally formulated in accordance with standard pharmaceutical practice as a pharmaceutical composition.

The compounds used in the instant invention can be administered in a therapeutically effective amount intravenously, subcutaneously, intramuscularly or any other method known to those skilled in the art (e.g., rectal, oral, parenteral). A suitable pharmaceutical composition used in this invention is one, which is made for sterile injection containing between 1 and 50% w/w of the compounds used in this invention.

With the co-administration of LF inhibitor and anthrax vaccine the relevant in vivo readout would be the development of anti-toxin, anti-capsule, or any other antibodies relevant to the anthrax vaccine in use.

For purposes of this invention, co-administration is any method utilizing the treatment therapies either simultaneously or sequentially within a short time after the first drug is administered. An example of co-administration is administering the vaccine (subcutaneously or intramuscularly) and the LF inhibitor (orally or intravenously) at the same time. The antibiotic could also be added at the same time as the LF inhibitor and vaccine. Another example would be to first administer the LF inhibitor (having a treatment schedule of at least 2 days, preferably 2 to 10 days) and then administer the vaccine sometime within the LF inhibitor treatment schedule. The antibiotic could be added at the same time the LF inhibitor is administered utilizing the same treatment schedule.

Suitable subjects for the administration of the formulation of the present invention include primates, man and other animals, particularly man and domesticated animals such as cats, rabbits and dogs.

The following non-limiting examples, given by way of illustration, is demonstrative of the present invention, that the compounds used in this invention are useful for treating anthrax and inhibiting lethal factor. The process for making the compounds discussed below is described in PCT application U.S. Ser. No. 03/16336, filed May 23, 2003 and incorporated herein by reference in its entirety.

Definition of terms are:
HOBT—hydroxybenzotriazole
DMF—dimethylformamide
DIEA—diisopropylethylamine
TMSONH2—O-trimethylsilylhydroxylamine
PyBOP—bnezotrizole-1-yl-oxy-tris-pyrrolidino-phosphonium hexafluorophosphate
TFA—trifluoroacetic acid
HPLC—high performance liquid chromatography
DCM—dichloromethane
EDC—1-(3-dimethylaminopropyl)-3-ethylcarbodiimide
THF—tetrahydrofuran
DIC—N,N′-diisopropylcarbodiimide
MDF—dimethylformamide
DMAP—4-dimethylaminopyridine
NMP—1-methyl-2-pyrrolidinone
EDTA—ethylenediaminetetraacetic acid

### Example 1

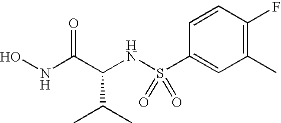

N-t-butoxy-2(R)-[(4-fluoro-3-methylphenylsulfonyl)]amino-3-methylbutyramide (1.8 g, 4.99 mmol) was dissolved in 75 ml of anhydrous dichloro-ethane containing ethanol (0.30 ml, 5 mmol) at 0° C. Hydrogen chloride gas was bubbled in for 30 min. The flask was closed with a septum and reaction mixture stirred for 2 days. After the solvent was removed on a rotavap, the residue was dissolved in methanol (1˜2 ml), and diluted with DCM (20 ml). The crystals formed were collected and washed with more DCM to give, after vacuum drying, N-hydroxy-2(R)-[(4-fluoro-3-methylphenylsulfonyl)]amino-3-methylbutyramide. NMR (500 MHz, CD₃OD) δ: 0.86 (d, 3H), 0.91 (d, 3H), 1.86 (m, 1H), 2.30 (d, 3H), 3.30 (d, 1H), 7.16 (t, 1H), 7.67 (m, 1H), 7.72 (m, 1H).

The starting material for example 1 was prepared as follows:

D-Valine (1.39 g, 11.9 mmol) was dissolved in 80 ml of dioxane/water (1:1) containing K₂CO₃ (3.3 g, 24 mmol). A solution of 4-fluoro-3-methylphenyl-sulfonylchloride (10 mmol) in dioxane (4 ml) was dropped in with good stirring. The reaction mixture was stirred at room temperature for 30 min. Ethylacetate (80 ml), 1N HCl (50 ml) was added. The organic layer was washed with 1N HCl 2 times, and extracted with 5% K₂CO₃ (3×25 ml). The combined base extracts was acidified and extracted with ethylaceate (80 ml). The organic layer was washed with brine (2×), dried over Na₂SO₄. The solvent was removed on rotavap, and residue tritrated with hexane. The resulting solid was dried to give 2(R)-[(4-fluoro-3-methylphenyl-sulfonyl)]amino-3-methylbutyric acid.

2(R)-[(4-Fluoro-3-methylphenylsulfonyl)]amino-3-methylbutyric acid (2.64 g, 9.12 mmol) was dissolved in DCM (30 ml), followed by addition of DIEA (3.18 ml, 2 eq.) and O-t-butylhydroxylamine hydrochloride (2.3 g, 2 eq.). EDC.HCl (2.1 g, 1.2 eq.) was then added portionwise as solid. More EDC (0.6, 0.5 eq.) was added after 40 min and the reaction was stirred for another 30 min. The solvent was removed on a rotavap at room temperature, and residue was partitioned with ethylacetate (80 ml), 1N HCl (50 ml). The organic layer was washed with 1N HCl, brine, and dried over Na₂SO₄. The crude product was flash column purified with 5% to 12% ethylacetate in DCM gradient solvent to give product N-t-butoxy-2(R)-[(4-fluoro-3-methylphenylsulfonyl)]amino-3-methylbutyramide as a white foam. TLC (1:10 ethylaceate:DCM) Rf 0.16. NMR (500 MHz, CD₃OD) δ: 0.89 (d, 3H), 0.90 (d, 3H), 1.08 (s, 9H), 1.86 (m, 1H), 2.30 (d, 3H), 3.44 (d, 1H), 7.18 (t, 1H), 7.70 (m, 1H), 7.77 (m, 1H).

### Example 2

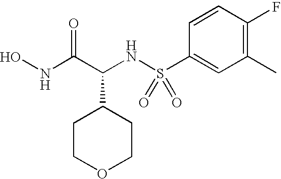

Example 2, N-hydroxy-2(R)-[(4-fluoro-3-methylphenylsulfonyl)]-amino-2-(4′-tetrahydropyranyl)-acetamide, was prepared from D-4′-tetrahydro-pyranylglycine in the same way as example 1. NMR (500 MHz, CD₃OD) δ: 1.19 (m, 1H), 1.34 (m, 1H), 1.40 (m, 1H), 1.74 (m, 1H), 1.80 (m, 1H), 2.32 (d, 3H), 3.31 (m, 2H), 3.37 (d, 1H), 3.90 (m, 2H), 7.18 (t, 1H), 7.65 (m, 1H), 7.72 (m, 1H).

### Example 3 to 144

Examples 3 to 144, found in Table 1, were made on solid phase and is illustrated as follows

Step 1. Resin functionalization

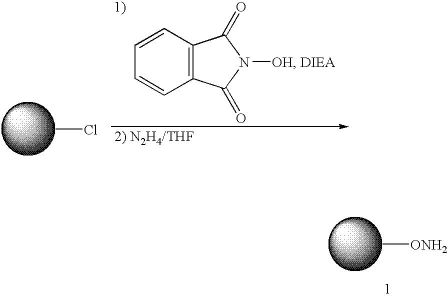

A solution of N-hydroxyphthalimide (2.8 g, 17 mmol), DIEA (3.0 ml, 17 mmol) in dichloromethane (30 ml) and DMF (15 ml) was added quickly to 4.39 g of 2-Chlorotrityl resin (1.1 mmol/g loading) in a frit fitted cartridge. The resin suspension was shaken intermittently and left on bench overnight. The resin was washed 5× with DMF, and then treated with a 40 ml of hydrazine solution (0.5 M in THF) for 2 hr. A large amount of white solid formed around the resin. It was washed with DMF-H₂O(1:1) 2×, DMF 4×. The hydrazine treatment was repeated once more for another 3 hours. The resin was washed with DMF-H₂O (1:1) 2×, DMF 4×, DCM 5×, dried in vacuum overnight to give 4.53 g of resin 1. The loading is about 1.0 mmol/g by weight change.

Step 2. Loading of Amino acid

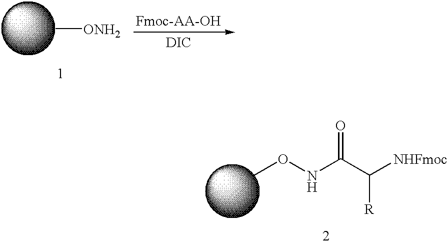

The O-anchored hydroxylamine resin 1, 500 mg (˜1.0 mmol/g loading), was swelled with DCM in a frit fitted cartridge and drained. A solution of Fmoc-D-allo-isoleucine (530 mg, 1.5 mmol, 3 eq.), DIC (0.120 ml, 0.75 mmol, 1.5 eq.) in 3 ml of DMF was added. The cartridge was shaken briefly and left on bench for 1 hr. Another dose of DIC (0.04 ml, 0.25 mmol, 0.5 eq.) was added. After another hour, the resin was washed with DMF 4×, DCM 4× and vacuum dried overnight to give resin 2. The approximate loading is 0.70 mmol/g by weight gain.

### Step 3

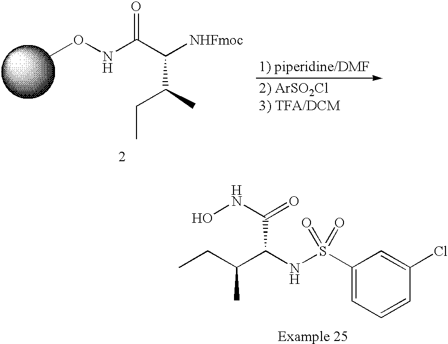

Resin 2, 150 mg, ˜0.7 mmol/g loading, was treated with 2 ml of piperidine/DMF (25%) for 2 hr. The resin was washed with MDF 3×, DCM 3×. A solution of DIEA (73 ul, 0.42 mmol, 4 eq.) in THF-DCM (1:1, 0.5 ml) containing DMAP (˜2 mg) was added to the resin, followed by a solution of 3-chlorophenylsulfonyl chloride (66 mg, 3 eq.) in THF-DCM (0.5 ml). After 3 hr, the resin was washed with DMF 3×, DCM 3×, and cleaved twice with 5% TFA/DCM (0.5 ml) for 30 min. The combined cleavage solution was evaporated, and the residue dissolved in CH₃CN:H₂O and purified on a reverse phase HPLC to give Example 25, N-hydroxy-2(R)-(3-chlorophenylsulfonyl)amino-3(S)-methylvaleric amide. NMR (500 MHz, CD₃OD) δ: 0.82 (d, d, 6H), 1.04 (m, 1H), 1.35 (m, 1H), 1.64 (m, 1H), 3.52 (d, 1H), 7.50 (t, 1H), 7.60 (d, 1H), 7.76 (d, 1H), 7.84 (m, 1H).

Table 1 lists structures of examples 3 to 144. As can be appreciated by the ordinary skilled artisan, Examples 4 to 144 were made, with some modification, in accordance with the description provided for example 3. Some compounds required a de-protection step (treatment with 50% TFA/DCM) after cleavage off the resin.

### Example 145

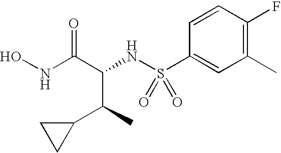

2-(R)-[(4-fluoro-3-methylphenyl)sulfonyl]amino-3-(S)-cyclopropyl-butyric acid (10 mg, 31 umol) was dissolved in DMF (0.3 ml) with HOBt (4.5 mg, 0.031 mmol), DIEA (11 ul, 0.062 mmol), O-trimethylsilylhydroxylamine (20 ul, 0.16 mmol). A solution of PyBOP (20 mg, 0.038 mmol) in DMF (0.3 ml) was added. The reaction was quenched after 30 min with CH₃CN:H₂O (1:1, 5% TFA) and passed through reverse phase HPLC to give, after lyophilization, N-hydroxy-2-(R)-[(4-fluoro-3-methylphenyl)sulfonyl]amino-3-(S)-cyclopropylbutyramide. NMR (500 MHz, CD₃OD) δ: −0.04 (m, 1H), 0.20 (m, 1H), 0.35 (m, 1H), 0.41 (m, 1H), 0.54 (m, 1H), 0.90 (d, 3H), 1.08 (m, 1H), 2.32 (d, 3H), 3.60 (d, 1H), 7.17 (t, 1H), 7.68 (m, 1H), 7.75 (m, 1H). MS: 331.1 (M+H⁺).

The starting material for example 145 was prepared as follows:

Methyl glycolate (10.4 g, 114 mmol), crotyl alcohol (100 ml, excess), was refluxed in the presence of K₂CO₃ (0.8 g) for 1 hr, during which time about 10 ml of the condensate was removed through a Dean-Stock trap. After diluting with hexane (100 ml), the solid was filtered through a short silica gel column (50 g), washed with 1:5 ethylacetate:hexane (250 ml). The combined filtrate and washings was concentrated to 100 ml, and was diluted again with hexane (100 ml), passed through silica gel column and washed. The solution was concentrated to ˜12.5 g of oil, which was vacuum distilled to give crotyl glycolate: 9.3 g (97° C./20 mmHg) as a mixture of cis:trans (1:10). NMR (500 MHz, CDCl₃) δ: 1.3 (m, 3H), 4.15 (s, 2H), 4.62 (d, 2H), 5.6 (m, 1H), 5.84 (m, 1H). cis isomer: 1.71 (m, 3H), the rest peaks overlaps with trans isomer.

The above made crotyl glycolate (9.3 g, 71 mmol) in THF (10 ml) was added slowly to a solution of LiN(TMS)₂ (200 ml, 1.0 M) in THF (200 ml) at −78° C. After 40 min at this temperature, trimethylsilyl chloride (25.5 ml, 200 mmol) was added. The cooling bath was removed and the reaction was stirred overnight. The reaction mixture was concentrated to ˜150 ml and diluted with ethylacetate (500 ml). This was washed with 2N HCl twice. The washings were back extracted with more ethylacetate. The combined organic layer was extracted with 5% K₂CO₃ 3×. The combined base solution was acidified with cold concentrated HCl, extracted with ethylacetate. The ethylacetate solution was washed with saturated NaCl, dried over Na₂SO₄. Evaporation of solvent and vacuum drying gave 2-hydroxy-3-methylpropen-4-enoic acid as a mixture of diastereomers. NMR (500 MHz, CD₃OD) for diastereomer 1 [(2R, 3S) and (2S, 3R)] δ: 1.02 (d, 3H), 2.60 (m, 1H), 4.05 (d, 1H), 5.02 (m, 1H), 5.09 (m, 1H), 5.87 (m, 1H); diasteteomer 2 [(2R, 3R) and (2S, 3S)] δ: 1.11(d, 3H), 2.6 (m, 1H), 4.03 (d, 1H), 5.0 (m, 1H), 5.09 (m, 1H), 5.80 (m, 1H). Diastereomeric ratio by NMR is about 7 to 1 with diasteromer 1 as the major.

The above made acid (8.5 g, 65 mmol) was dissolved in dry DMF (100 ml) and DIEA (16 ml, 91 mmol). Methyl iodide (11.7 ml, 85 mmol) was added. This was stirred for 15 hr, and diluted with ethylacetate (500 ml), washed with 0.1N HCl 3×, brine 2×, dried over Na₂SO₄. Evaporation of solvent left Methyl 2-hydroxy-3-methylpenten-4-enoic ester. NMR (500 MHz, CD₃OD) for diastereomer 1 [(2R, 3S) and (2S, 3R)] δ: 1.02 (d, 3H), 2.55 (m, 1H), 3.70 (s, 3H), 4.04 (d, 1H), 5.02 (m, 1H), 5.06 (m, 1H), 5.81 (m, 1H); diasteteomer 2 [(2R, 3R) and (2S, 3S)] δ: 1.08 (d, 3H), 2.58 (m, 1H), 3.70 (s, 3H), 4.07 (d, 1H), 5.00 (m 1H), 5.06 (m, 1H), 5.80 (m, 1H).

The above made methyl ester (2.9 g, 20 mmol) was dissolved in dry DCM (100 ml) with diiodomethane (8.1 ml, 100 mmol), and cooled to 0° C. A solution of diethylzinc (100 ml, 1.0 M in hexane) was added. The cooling bath was removed and the mixture was stirred under nitrogen for 3 days. A solution of NH₄Cl was added to quench the reaction. The organic layer was washed with HCl 2×, brine 2×, and dried over Na₂SO₄ Evaporation of solvent left oil containing 70% of product methyl 2-hydroxy-3-cyclopropylbutyrate and 30% of starting material. It was used without further purification.

A solution of the above made ester (3 g, 20 mmol), pyridine (2.0 ml, 24 mmol) in dry DCM (10 ml) was slowly added to a stirred solution of Tf₂O (4.0 ml, 24 mmol) in DCM (100 ml) at 0° C. After 1 hr at 0° C., water was added to quench the reaction. This was then washed with dilute HCl (0.1 N), brine, and dried over Na₂SO₄ Evaporation of solvent gave 5.3 g of triflate as an oil. This was stirred with NaN₃ (2.4 g, 36 mmol) in DMF (80 ml) for 15 hr. The reaction mixture was diluted with ethylacetate (400 ml), washed with dilute HCl 3×, brine 2×, dried over Na₂SO₄ Evaporation of solvent Ifet 2.96 g of oil. Flash column chromatography though silica gel, eluting with 5% ether in hexane gave methyl 2-azido-3-cyclopropyl-butyrate as a colorless oil. The desired diastereomer 1 [(2R, 3S) and (2S, 3R)] can be isolated through preparative reverse phase HPLC eluting with CH₃CN:H₂O gradient solvent. NMR (500 MHz, CDCl₃) for diastereomer 1 [(2R, 3S) and (2S, 3R)] δ: 0.04 (m, 1H), 0.18 (m, 1H), 0.48 (m, 2H), 0.74 (m, 1H), 1.09 (d, 3H), 1.35 (m, 1H), 3.80 (s, 3H), 3.92 (d, 1H).

The above isolated azide [(2R, 3S) and (2S, 3R)] diastereomer (400 mg, 2.2 mmol) was dissolved in MeOH (10 ml), cooled in a water bath at 20° C. Stannous chloride (860 mg, 4.4 mmol) waw added. This was stirred for 15 hr. To the reaction mixture was added with dioxane (10 m10), K₂CO₃ (1.5 g 10.1 mmol)/H₂O (10 ml). The solid was filtered, washed with dioxane (5 ml). To the combined filtrate and washings was added a solution of 4-fluoro-3-methylphenylsulfonyl chloride (560 mg, 2.4 mmol) in dioxane (5 ml). About 30 min later, the reaction was acidified with HCl to pH 3, diluted with CH₃CN:H₂O. The product was isolated through preparative reverse phase HPLC (repeated injections) to Methyl 2-(4-fluoro-3-methylphenylsulfonamido)-3-cyclopropylbutyrate. Further separation through Chiralpk column AD eluting with 7% EtOH in heptane gave two enantiomers, with the desired isomer 1 (2R, 3S) eluted out first. NMR (500 MHz, CD₃OD) δ: 0.01 (m, 2H), 0.39 (m, 2H), 0.62 (m, 1H), 1.01 (d, 3H), 1.19 (m, 1H), 2.312 (d, 3H), 3.23 (s, 3H), 3.90 (d, 1H), 7.18 (t, 1H), 7.68 (m, 1H), 7.73 (m, 1H).

Methyl 2(R)-[(4-fluoro-3-methylphenyl)sulfonyl]amino-3-(S)-cyclopropyl-butyric ester (20 mg, 0.061 mmol) was dissolved in MeOH (0.2 ml), followed by addition of LiOH (8 mg, excess)/H₂O (0.15 ml). After 2 hr the reaction was acidified with 1.5 ml of CH₃CN:H₂O (1:1, 5% TFA) and chromatographed with reverse phase HPLC to give 2-(R)-(4-fluoro-3-methylphenyl-sulfonamido)-3-(S)-cyclopropylbutyrc acid. NMR (500 MHz, CD₃OD) δ: −0.01 (m, 1H), 0.15 (m, 1H), 0.40 (m, 2H), 0.65 (m, 1H), 1.02 (d, 3H), 1.22 (m, 1H), 2.31 (d, 3H), 4.83 (d, 1H), 7.16 (t, 1H), 7.69 (m, 1H), 7.75 (m, 1H).

### Example 146

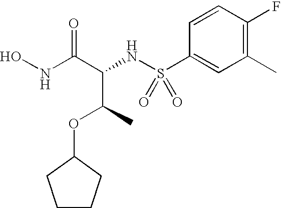

2(R)-[(4-Fluoro-3-methylphenyl)sulfonyl]amino-3(R)-cyclopentoxylbutyric acid (11 mg, 0.03 mmol) was dissolved in DMF (200 ul) with DIEA (12 ul, 0.12 mmol), HOBt (8 mg, 0.06 mmol), and TMSONH₂ (10 ul, 0.08 mmol). A solution of PyBOP (31 mg, 0.06 mmol) in DMF (100 ul) was added. The reaction was quenched after 20 min with 5% TFA/H₂O, and product isolated from reverse phase HPLC to give, after lyophilization, N-hydroxy-2(R)-[(4-fluoro-3-methylphenyl)sulfonyl]amino-3(R)-cyclopentoxylbutyramide. NMR (500 MHz, CD₃OD) δ: 0.97 (d, 3H), 1.44-1.68 (m, 8H), 2.32 (d, J_{H-F}, 3H), 3.61 (d, 1H), 3.72 (m, 1H), 3.67 (m, 1H), 7.18 (m, 1H), 7.70 (m, 1H), 7.76 (m, 1H).

The starting material for example 146 was prepared as follows:

N-Trityl-D-threonine benzyl ester (2.5 g, 5.5 mmol), TEA (2.8 ml, 20 mmol) were dissolved in 100 ml of dry toluene at −50° C. A solution of sulfuryl chloride (800 ul, 8 mmol) in toluene (20 ml) was added in 15 min. The reaction was allowed to warm up to r.t. Ethylacetate (100 ml) was added and this was washed with sat. NaCl, dried over Na₂SO₄. The product was crystallized in MeOH (10 ml) to give benzyl N-trityl-3(S)-methylaziridine-2(R)-carboylate. NMR (500 MHz, CDCl₃) δ: 1.37 (d, 3H), 1.64 (m, 1H), 1.95 (d, 1H), 5.15 (d, J=12 Hz, 1H), 5.28 (d, J=12 Hz, 1H), 7.19˜7.28 (m, 12H), 7.33˜7.36 (m, 1H), 7.36˜7.39 (m, 3H), 7.51˜7.54 (m, 4H).

Benzyl N-trityl-3(S)-methylaziridine-2(R)-carboxylate, (2.13 g, 4.92 mmol) was dissolved in 20 ml of MeOH:DCM (1:1) at 0° C., followed by addition of TFA (20 ml). After stirring at room temperature for 1 hr, the excess reagent and solvent were removed on rotavap (T<25° C.). The residue was partitioned with DCM (50 ml) and H₂O (100 ml). The aqueous phase was washed once with DCM, and pH was adjusted to basic with NaHCO₃ extracted with ethylacetate, and dried over Na₂SO₄. Removal of solvent left 650 mg of Benzyl 3(S)-methylaziridine-2(R)-carboxylate. This was dissolved in DMF (15 ml) at 0° C. TEA (2.1 ml, 15 mmol) was added, followed by Boc₂O (1.64 g, 7.5 mmol). The reaction was stirred at room temperature overnight. Ethylacetate (100 ml), H₂O (100 ml) were added, and the organic layer was washed with 10% citric acid twice, brine, and dried over Na₂SO₄. The crude product was flash column chromatographed, eluting with 5%˜10% EA/hexane gradient solvent containing 0.1% TEA, to give benzyl N-Boc-3(S)-methylaziridine-2(R)-carboxylate. NMR (500 MHz, CD₃OD) δ: 1.21(d, 3H), 1.44 (s, 9H), 2.82 (m, 1H), 3.21 (d, 1H), 5.2 (q, 2H), 7.30˜7.38(m, 5H).

Benzyl N-Boc-3(S)-methylaziridine-2(R)-carboxylate (50 mg, 0.17 mmol), cyclopentyl alcohol (0.5 ml, 5.5 mmol) were dissolved in DCM (0.5 ml), followed by a few drops of BF₃.Et₂O. This was stirred at r.t. for 10 hr. The solvent was removed, and the residue purified through a reverse phase HPLC. The product was collected and treated with 50% TFA/DCM to give benzyl 2(R)-amino-3(R)-cyclopentoxylbutyrate triflruoroacetate. NMR (500 MHz, CD₃OD) δ: 1.28 (d, 3H), 1.4˜1.7 (m, 8H), 3.92 (m, 1H), 4.06 (d, 1H), 4.14 (dq, 1H), 5.26 (d, J=12 Hz, 1H), 5.31 (d, J=12 Hz, 1H), 7.38 (m, 3H), 7.43 (m, 2H).

Benzyl 2(R)-amino-3(R)-cyclopentoxylbutyrate triflruoroacetate (63 mg, 0.16 mmol), DIEA (174 ul, 1.0 mol), DMAP (1 mg) were dissolved in dioxane (2 ml), followed by slow addition of a solution of 4-fluoro-3-methylphenylsulfonyl chloride (˜0.33 mmol) in dioxane (1 ml). After 15 min, the reaction was quenched with 5% TFA/H₂O, and purified through reverse phase HPLC to give benzyl 2(R)-[(4-Fluoro-3-methylphenyl)sulfonyl]amino-3(R)-cyclopentoxylbutyrate. The benzyl ester protection group was removed by hydrogenation in MeOH:EA (1 ml) with 10% Pd/C (2 mg) overnight to give 2(R)-[(4-Fluoro-3-methylphenyl)-sulfonyl]amino-3(R)-cyclopentoxylbutyric acid.

With some modification known to those skilled in the art, Examples 147 to 153 of Table 2 were made in accordance with Example 146.

### Administration

Vaccination would occur while patient was being treated with LF inhibitor intravenously, intramuscularly, or orally. This administration of the LF inhibitor would allow a normal, effective presentation of antigens present in the vaccine by dendritic or other antigen presenting cells. This presentation of antigen would elicit T and B cell responses characteristic of host immunity. Antibiotic treatment would be administered as soon as possible after a patient was suspected to have been exposed to anthrax. Specific vaccination and/or antibiotic administration would occur in accordance with the instructions provided by the manufacturer of the drug(s).

### Assay for Determining Lethal Factor Inhibition

The assay below is disclosed in Cummings et al., PNAS, May 14, 2002, vol. 99, no. 10, page 6603-6606 and PCT Application US03/05552, filed Feb. 21, 2003 (U.S. Patent application Ser. No. 60/359,707, filed Feb. 25, 2002), incorporated herein by reference in their entirety. It is used to determine lethal factor inhibition after being reacted with a compound believed to be an inhibitor of lethal factor.

Lethal factor inhibitor compounds can be used to further study lethal factor activity, and those inhibitory compounds having appropriate pharmacological properties can be used to help treat or prevent Anthrax. Appropriate pharmacological properties include efficacy, metabolism and absence of unacceptable side effects.

High throughput screening for lethal factor inhibitors can be used to screen large number of compounds to identify those affecting lethal factor activity. High throughput screening is facilitated by an assay that is readily automated and utilizes low levels of purified enzyme.

Lethal factor substrates can be used in methods measuring Bacillus anthracis lethal factor activity and the effect of a compound on such activity. Such methods involve incubating a lethal factor substrate described herein with Bacillus anthracis lethal factor using an incubation medium where the Bacillus anthracis lethal factor is active, and can include the presence of a compound being tested. Cleavage of the substrate can be detected as a measure of Bacillus anthracis lethal factor activity or the effect of a compound on lethal factor activity. Measuring can be qualitative or quantitative. The lethal factor enzyme binding assay IC50 results for the compounds used in this invention range from 15 uM or less. Specifically the IC50 for N-hydroxy-2(R)-[(4-fluoro-3-methylphenylsulfonyl)]amino-3-methylbutyramide and N-hydroxy-2(R)-[(4-fluoro-3-methylphenylsulfonyl)]amino-2-(4′-tetrahydropyranyl)-acetamide are 0.13 uM and 0.06 uM respectively.

1. (canceled)

2. A method for treating anthrax or inhibiting lethal factor by co-administration of a vaccine and a lethal factor inhibitor of structural formula I: 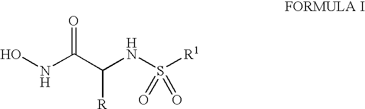
or a pharmaceutically acceptable salt or mixture thereof, wherein,
R¹ is selected from the group consisting of C₆₋₁₀ aryl substituted with 2 groups of R^{a}; each R^{a} is independently selected from the group consisting of C₁₋₆ alkyl and halogen; and
R is selected from the group consisting of C₃₋₁₀ heterocycloalkyl and C₅₋₁₀ heteroaryl, wherein said vaccine is any type of anthrax vaccine.

3. The method according to claim 2, wherein the vaccine is a protective antigen based vaccine, including a capsule-based or a conjugate of protective antigen and capsule-based vaccines.

4. The method according to claim 3, wherein the vaccine is selected from the group consisting of anthrax vaccine, protective antigen vaccine, Bacillus anthracis live spore vaccine, protective antigen toxoid vaccines, protective antigen producing live vaccines, and recombinant anthrax toxin vaccine.

5. A method of inhibiting lethal factor and/or treating anthrax comprising the co-administration of a lethal factor inhibitor, a vaccine and one or more known drugs selected from beta-lactams, aminoglycosides, inhibitors of beta-lactamase, renal tubular blocking agents and inhibitors of metabolising enzymes, N-acylated amino acids, wherein said lethal factor inhibitor is a compound of structural formula I: 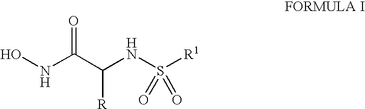
or a pharmaceutically acceptable salt or mixture thereof, wherein,
R¹ is selected from the group consisting of C₆₋₁₀ aryl substituted with 2 groups of R^{a}; each R^{a} is independently selected from the group consisting of C₁₋₆ alkyl and halogen; and
R is selected from the group consisting of C₃₋₁₀ heterocycloalkyl and C₅₋₁₀ heteroaryl.

6. A method according to claim 5, wherein the known drugs are selected from the group consisting of imipenem, meropenem, vancomycin, cilastatin, cefoxitin, penicillin, clavulanic acid, doxycycline, tetracycline, chloramphenicol, erythromycin, cefazolin, rifampin, clindamycin, clarithromycin, azithromycin, ceftriaxone, sulfamethoxazole, and trimethoprim, probenecid, tetracycline, ciprofloxacin, and norfloxacin or a mixture thereof, wherein when imipenem is used as a drug it is used in combination with cilastatin as PRIMAXIN®, the vaccine is selected from the group consisting of anthrax vaccine, protective antigen vaccine, Bacillus anthracis live spore vaccine, protective antigen toxoid vaccines, protective antigen producing live vaccines, and recombinant anthrax toxin vaccine and the lethal factor inhibitor is a compound selected from the group consisting of:
N-t-butoxy-2(R)-[(4-fluoro-3-methylphenylsulfonyl)]amino-2-(4′-tetrahydropyranyl)-acetamide;
N-hydroxy-2(R)-[(4-fluoro-3-methylphenylsulfonyl)]amino-2-(4′-tetrahydropyranyl)-acetamide;
and pharmaceutically acceptable salts or mixtures thereof.

7-9. (canceled)

10. The method of claim 2, wherein said lethal factor inhibitor is N-hydroxy-2(R)-[(4-fluoro-3-methylphenylsulfonyl)]amino-2-(4′-tetrahydropyranyl)-acetamide or a pharmaceutically acceptable salt thereof.

11. The method of claim 2, wherein said method is performed on a patient infected with anthrax.

12. The method of claim 11, wherein said lethal factor inhibitor is N-hydroxy-2(R)-[(4-fluoro-3-methylphenylsulfonyl)]amino-2-(4′-tetrahydropyranyl)-acetamide or a pharmaceutically acceptable salt thereof.
